(19) United States
(12) Patent Application Publication
(10) Pub. No.: US 2008/0234730 A1
(43) Pub. Date: Sep. 25, 2008

(54) Fixation Devices and Method of Repair

(75) Inventors: Nicholas Cotton, Westborough, MA (US); Rebecca A. Blough, Cumberland, RI (US); Melissa J. Egan, Plympton, MA (US); Horacio Montes de Oca Balderas, York (GB); Malcolm Brown, Otley (GB); Michael Hall, Linthrope (GB)

Correspondence Address: NORMAN F. HAINER, JR.;SMITH & NEPHEW, INC., 150 MINUTEMAN ROAD, ANDOVER, MA, 01801, US

(73) Assignee: Smith & Nephew, Inc., Andover, MA (US)

(21) Appl. No.: 12/053,264

(22) Filed: Mar. 21, 2008

Related U.S. Application Data
(60) Provisional application No. 60/896,945, filed on Mar. 26, 2007.
(60) Provisional application No. 60/896,520, filed on Mar. 23, 2007.

(51) Int. Cl. A61B 17/04; A61B 17/56

(52) U.S. Cl. 606/232; 606/304

(57) ABSTRACT

In one aspect, the present disclosure relates to a surgical device including an anchor body having an opening, the anchor body having a copolymer composition including polylactide-co-glycolide and calcium carbonate, wherein the calcium carbonate comprises more than 30% but less than 40% of the weight of the composition; and a flexible member passing through the opening, wherein deformation of the device occurs at body temperature. The present disclosure also relates to an oriented polymer material having a copolymer composition including a polylactide-co-glycolide and calcium carbonate, the calcium carbonate comprising more than 30% but less than 40% of the weight of the composition, wherein the material changes shape upon introduction to an environment having a temperature that is lower than a relaxation temperature of the material. A method of repairing soft tissue and other surgical devices are also disclosed.

### CROSS-REFERENCE TO RELATED APPLICATIONS

This application claims priority to U.S. Provisional Application No. 60/896,945, filed on Mar. 26, 2007, and U.S. Provisional Application No. 60/896,520, filed on Mar. 23, 2007. The disclosures of each of these applications are incorporated herein by reference in their entirety.

### BACKGROUND OF THE INVENTION

1. Field of the Invention

The present disclosure relates generally to soft tissue fixation and specifically to devices and methods for improving soft tissue fixation to bone.

2. Related Art

Soft tissues, such as ligaments and tendons, can become torn or detached from bone. The tear or detachment can be repaired by inserting a surgical device, such as an anchor having an attached suture, into bone, and knotting the suture to secure the soft tissue to the bone. Once placed in bone, these surgical devices are required to exhibit certain fixation strength for a certain time to enable the soft tissue to heal back to the bone. Currently, there is a certain limitation to the size and bone quality that these devices can be used in to give the minimum amount of fixation required to anchor the soft tissue back to the bone. Therefore, a surgical device that can function in a wide range of bone qualities is needed.

### SUMMARY OF THE INVENTION

In one aspect, the present disclosure relates to a surgical device including an anchor body having an opening, the anchor body having a copolymer composition including polylactide-co-glycolide and calcium carbonate, wherein the calcium carbonate comprises more than 30% but less than 40% of the weight of the composition; and a flexible member passing through the opening. Deformation of the device occurs at body temperature. In an embodiment, the opening includes a through hole. In a further embodiment, the anchor body includes screw threads and is configured for rotary advancement into a target tissue. In yet another embodiment, the anchor body includes circumferential ribs and is configured for axially oriented advancement into a target tissue. In an embodiment, the surgical device is injection molded. In another embodiment, deformation of the device occurs at about 37° C. In yet another embodiment, the device is bioabsorbable.

In another aspect, the present disclosure relates to a method for repairing a soft tissue. The method includes placing a surgical device, having a flexible member coupled thereto, into bone; passing the flexible member through a soft tissue located adjacent to the bone; and tying the flexible member to secure the soft tissue to the bone. Deformation of the device occurs at body temperature after placement of the device in the bone. The surgical device includes a copolymer composition having polylactide-co-glycolide and calcium carbonate, wherein the calcium carbonate comprises more than 30% but less than 40% of the weight of the composition. In an embodiment, the surgical device includes a suture anchor. In another embodiment, deformation of the device provides an increase in fixation of the device to the bone. The increase in fixation includes an increase in fixation strength of the device of between of about 50% to about 200%. In yet another embodiment, deformation of the device provides an increase in width of the device and a decrease in length of the device.

In yet another aspect, the present disclosure relates to an oriented polymer material that changes shape upon introduction to an environment having a temperature that is lower than a relaxation temperature of the material. The oriented polymer material has a copolymer composition including a polylactide-co-glycolide and calcium carbonate, the calcium carbonate comprising more than 30% but less than 40% of the weight of the composition. In yet another embodiment, the polylactide-co-glycolide includes poly (D,L-lactide-co-glycolide). In a further embodiment, the copolymer includes at least one mobile polymer. In another embodiment, the copolymer further includes at least one rigid polymer. In yet another embodiment, the copolymer further includes at least one mobile polymer and one rigid polymer. The mobile polymer includes polyethylene glycol and the rigid polymer is selected from a group including D-lactide, L-lactide, and D,L lactide. In a further embodiment, the polymer material includes a porogen, such as sodium chloride.

The environment includes a temperature of the environment is about 37° C. In an embodiment, the temperature of the environment is body temperature. The relaxation temperature is about 500 and the polymer material includes a fixation strength of above 500 N.

In a further aspect, the disclosure also relates to a surgical device including a copolymer composition having a polylactide-co-glycolide and a porogen. In an embodiment, the surgical device includes an oriented polymer material. In another embodiment, the oriented polymer material is made by a process selected from a group including die drawing, hydrostatic extrusion, and roll drawing. In yet another embodiment, the porogen includes sodium chloride. In a further embodiment, the porogen is selected from a group including lithium bromide, lithium iodide, calcium chloride, sodium iodide, magnesium sulphate, and calcium sulphate. In yet a further embodiment, the surgical device is selected from a group including pins, rods, nails, screws, plates, anchors, and wedges.

In yet a further aspect, the present disclosure relates to a surgical device including a first component having a shaft, a second component coupled to the first component, and a flexible member coupled to the shaft, wherein the first component is an injection molded component and the second component includes an oriented polymer material. In an embodiment, the flexible member is coupled to the shaft via an eyelet, the eyelet being coupled to the shaft. In another embodiment, the flexible member is coupled to the shaft via an opening in the shaft. In yet another embodiment, the first component includes a copolymer composition having polylactide-co-glycolide and calcium carbonate and the second component includes a copolymer composition having polylactide-co-glycolide and a porogen. In a further embodiment, the oriented polymer material is made by a process selected from a group including die drawing, roll drawing, and hydrostatic extrusion.

In another aspect, the present disclosure relates to a surgical device comprising a first component including a shaft and a second component coupled to the first component, wherein the first component includes a copolymer composition having polylactide-co-glycolide and calcium carbonate and the second component includes a copolymer composition having polylactide-co-glycolide and a porogen.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the disclosure. In the drawings:

FIG. 1 shows a first embodiment of the fixation device of the present disclosure.

FIG. 2 shows a second embodiment of the fixation device of the present disclosure.

FIG. 3 shows a method of repairing a tissue using a fixation device of the present disclosure.

FIG. 4A shows a fixation device of the present disclosure after the device has been inserted into bone.

FIG. 4B shows a fixation device of the present disclosure after the device has deformed.

FIG. 5 shows a measurement of the change in width of the device after the device has been inserted into bone.

FIG. 6 shows a measurement of the change in length of the device after the device has been inserted into bone.

FIG. 7 shows the fixation strength of the device after the device has been inserted into bone having a density of 20 pcf.

FIG. 8 shows the fixation strength of the device after the device has been inserted into bone having a density of 10 pcf.

FIG. 9 shows the dynamic mechanical thermal data of a polymer material of the present disclosure.

FIG. 10 shows a measurement of the increase in weight and diameter of a polymer rod after being placed at body temperature.

FIGS. 11A-11B show alternative fixation devices of the present disclosure.

### DETAILED DESCRIPTION OF THE EMBODIMENTS

The following description of the preferred embodiment(s) is merely exemplary in nature and is in no way intended to limit the disclosure, its application, or uses.

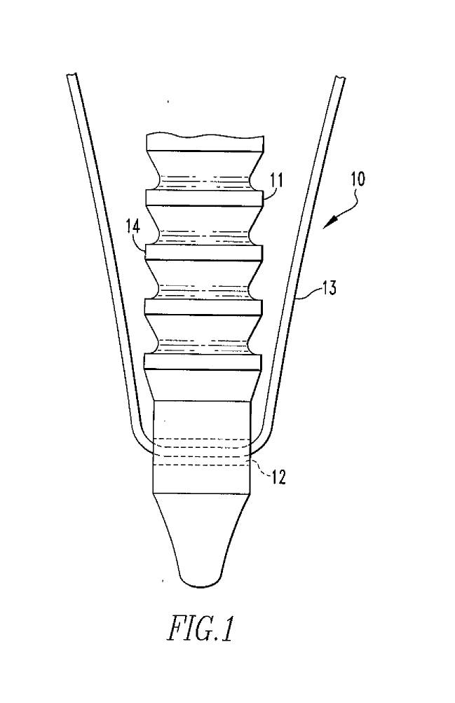

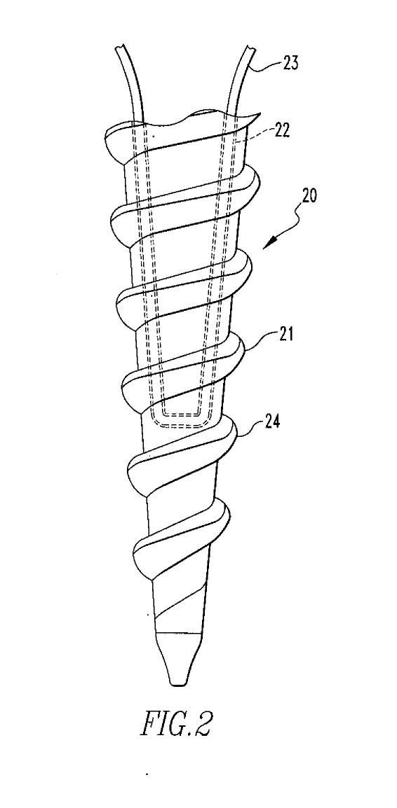

FIGS. 1 and 2 show first and second embodiments of a surgical device 10,20 of the present disclosure. Both figures show an anchor body 11,21 including an opening 12,22 and a flexible member 13,23, such as a suture, passing through the opening 12,22. The openings 12,22 in both anchor bodies 11,21 are through holes. However, the sutures 13,23 could be coupled to the devices 10,20 in other manners known to one of ordinary skill in the art. The device 10 shown in FIG. 1 includes circumferential ribs 14 along its length and is configured for axially oriented advancement into a target tissue. The anchor 10 is usually inserted into a bone by first creating an opening in the bone and then pounding the anchor 10 into the bone. The device 20 shown in FIG. 2 includes screw threads 24 along its length and is configured for rotary advancement into a target tissue. The anchor 20 is usually inserted into a bone by first creating an opening in the bone and then screwing the anchor 20 into the bone.

Both of the devices 10,20 shown in FIGS. 1 and 2 include a polymer composition containing a copolymer and a filler material. For example, the composition may include a copolymer that includes lactic acid and/or glycolic acid monomers and a filler such as calcium carbonate (e.g., about 30-40% CaCO₃ by weight (i.e., by weight of the composition as a whole).

In specific embodiments, the copolymer can be poly(lactide-co-glycolide) (PLGA), with a lactide:glycolide ratio of about 85.15 and the filler can be calcium carbonate. The compositions of the disclosure may be amorphous (i.e., they can be compositions in which the polymer chains are not ordered) or semi-crystalline (i.e., compositions in which there is some order to the polymer chains). In one embodiment, the disclosure features a biocompatible (i.e., substantially non-toxic) composition that includes a filler such as calcium carbonate and a copolymer formed from lactic acid monomers and glycolic acid monomers. The filler (e.g., calcium carbonate) can constitute more than 30% but less than 40% of the weight of the composition, regardless of the composition's form, the copolymer selected, or the inclusion of other components (e.g., a therapeutic agent, as described below).

For example, the filler (e.g., calcium carbonate) can constitute more than 30% but less than about 34%; more than 30% but less than about 35%; or about 36% to less than 40% of the weight of the composition. The filler can constitute more than 30%; about 31%; about 32%; about 33%; about 34%; about 35%; about 36%; about 37%; about 38%; about 39%; or an amount therein between (e.g., an amount between 31 and 32%; an amount between 32 and 33%; and so forth). Where calcium carbonate is used, it can have the crystalline structure of calcite, and it may be present as calcium carbonate particles of a substantially uniform size (e.g., a majority of the calcium carbonate particles can be about 0.1-0.5; 0.5-2.5; 2.5-5.0; 5.0-7.5; or about 7.5-10.0 μm in size (size being measured across the particles' largest diameter)). Alternatively, the filler particles can vary in size (e.g., ranging in size in a uniform or non-uniform way from about 0.01 μm to about 10.0 μm).

Other fillers that may be used include calcium carbonate, calcium hydrogen carbonate, calcium phosphate, dicalcium phosphate, tricalcium phosphate, magnesium carbonate, sodium carbonate, hydroxyapatite, bone, phosphate glass, silicate glass, magnesium phosphate, sodium phosphate, barium sulphate, barium carbonate, zirconium sulphate, zirconium carbonate, zirconium dioxide, bismuth trioxide, bismuth oxychloride, bismuth carbonate, tungsten oxide, or any combination thereof.

Any of the fillers, including CaCO₃, can be combined with a PLGA copolymer in which the lactic acid monomers are in the L-form or the D-form, or are a mixture of the L- and D-forms. More specifically, the copolymer can be poly(dl-lactide-co-glycolide). The ratio of lactic acid and glycolic acid monomers within the polymer may also vary. For example, the copolymer may contain from about 50:50 lactide:glycolide units to about 90:10 lactide:glycolide units (e.g., about 85:15 lactide:glycolide units). It will be understood by one of ordinary skill in the art that these ratios may, and often do, vary due to manufacturing limitations. For example, the ratio may vary by about +5%. Thus, it is to be understood that all references herein to the ratio of polymer units encompasses copolymers in which that ratio varies to an expected extent.

In a specific embodiment, the composition includes (and may include only) a copolymer of lactide and glycolide units and more than 30% but less than 40% calcium carbonate by weight. In another specific embodiment, the composition includes (and may include only) poly(lactide-co-glycolide) at 85:15 lactide:glycolide units and about 20-50% calcium carbonate by weight (e.g., about 20-30% (e.g., 25%), 30-40%, 40-50% (e.g., 45%), 30-34%, 35%, or 36-40%). Regardless of the precise components or their amounts, the copolymer may be amorphous or semi-crystalline and the filler (e.g. CaCO₃) and the copolymer (e.g., PLGA) may form a substantially homogeneous mixture (e.g., the filler can be evenly or about evenly distributed within the copolymer). Thus, the composition of the device, as a whole, fashioned from a substantially homogeneous mixture may also be homogeneous (e.g., the composition of a device at the proximal and distal ends can be substantially indistinguishable in content).

The compositions described herein may, but do not necessarily, contain one or more additional components, which may be bioactive agents (e.g., therapeutic agents). Examples of bioactives include any substance, such as a therapeutic agent or enzyme whose controlled, continuous release occurs over a period of time (e.g., some or all of the degradation period of the polymer) is desired. In an embodiment, the bioactive agent is hydrophobic, i.e. does not readily dissolve in water. The bioactive agent may be a protein, such as a degradation enzyme, cytokine or cytokine inhibitor, or a growth factor. For example, the compositions may contain a growth factor, including growth factors such as those from the fibroblast growth factor family, transforming growth factor family, epidermal growth factor (EGF), insulin-like growth factor-1 (IGF-1), thyroid-derived chondrocyte stimulating factor (TDCSF), and transforming growth factor-beta (TGF-.beta) platelet derived growth factor family that act as chemoattractants and/or growth stimulators, a hormone such as human growth hormone, an antibiotic, an antiviral agent, an antifungal agent, an anti-inflammatory agent, an inflammatory mediator such as an interleukin, tumor necrosis factor, a prostaglandin, nitric oxide, an analgesic agent, an osteogenic factor such as a bone morphogenetic protein, or a matrix molecule such as hyaluronan.

Other agents include angiogenic factors, which are capable of directly or indirectly promoting angiogenesis. Examples include angiogenic peptide growth factors in autologous, xenogenic, recombinant, or synthetic forms (e.g., a member of the vascular endothelial growth factor family). Further examples are blood clot breakdown products, such as thrombin and heparin including autologous, allogeneic, xenogeneic, recombinant and synthetic forms of these materials.

Compositions based around butyric acid, including butyric acid (butanoic acid, C₄H₈O₂) and butyric acid salts, including sodium, potassium, calcium, ammonium and lithium salts, α-monobutyrin (1-glycerol butyrate; 1-(2,3 dihydroxypropyl) butanoate; C₇H₁₄O₄) and hydroxybutyrate can also be incorporated. Where the bioactive or therapeutic agent is a polypeptide, one may incorporate the polypeptide in its naturally occurring form or a fragment or other mutant thereof that retains sufficient biological activity to confer a benefit on the patient to whom it is administered. The polypeptides may be autologous in the sense that, where the recipient is a human patient, the polypeptide may have the sequence of a human polypeptide or a biologically active fragment or other mutant thereof. Alternatively, or in addition, the additional component may be a nutraceutical, such as a vitamin or mineral.

The bioactive material is included in an amount that is therapeutically effective for the organism (e.g., a human patient) in question. Inclusion of one or more bioactive materials may, for example, increase the rate of tissue repair, decrease the risk of infection, or otherwise aid the healing or post-operative process. The release of the bioactives may be controlled through the relaxation rate of the polymer material, as will be further described below.

The manufacture of the devices 10,20 of FIGS. 1 and 2 can be carried out in steps that include the following: (a) providing a filler (e.g., calcium carbonate); (b) providing a copolymer (e.g. a copolymer formed from lactic acid monomers and glycolic acid monomers); (c) combining the filler and the copolymer to produce a composition in which the amount of the filler constitutes about 20-50% of the composition (e.g., more than 30% and less than 40% of the composition (e.g., about 35%)); and (d) processing the composition to produce a device 10,20. A further step of forming suture holes in the device 10,20 and inserting a suture into the holes may be added. The suture holes may be formed by drilling or by some other method of forming the holes. Optionally, the suture holes may be integral with the mold design and would be present in the device 10,20 after processing. An even further step of sterilizing the device by, for example, exposing it to radiation (e.g., gamma radiation), treating it with gases (e.g., chemical sterilization such as exposure to ethylene oxide gas), exposing it to heat (e.g., from steam, as in autoclaving), or exposing it to an electronic beam (e beam), or light (e.g., white light) could be added. Methods of sterilizing devices are known in the art, and one of ordinary skill in the art may select methods appropriate for a given device.

Optionally, the filler and copolymer may be combined with a bioactive agent (e.g., a therapeutic agent) including, but not limited to, any of those described herein. The therapeutic agent may be mixed or otherwise combined with the copolymer and filler or it can be added to the surface of the device or otherwise localized within the device.

For the purposes of this disclosure, the devices 10,20 may be formed by an injection molding process. Injection molding parameters are selected to give molded-in stresses to the polymer chains present in the polymer composition of the devices 10,20. Critical parameters of the injection molding process include, but are not limited to, injection speed, mold temperature, packing pressure, and gate geometry. Exact molding conditions are dependent on material specifications and the inherent properties of the material. Generally, the injection speed is decreased, keeping melt and mold temperatures constant until the slowest injection speed is determined while still filling the mold. The mold temperature is similarly decreased to as low as possible while still filling the mold. The packing pressure is increased until material leakage from the mold becomes unacceptable and the gate geometry is kept as small as possible to give a good mold fill and practicable fill times.

In the present disclosure, molded articles, such as the devices 10,20 in FIGS. 1 & 2, to which a definite shape has been imparted by injection molding, are deformed into a different shape once they are placed in the body. Upon insertion of the devices 10,20 into the body, it is believed that the above mentioned molded-in stresses are relaxed due to the absorption of water and thermal energy by the devices 10,20. The absorption of water and thermal energy, via diffusion, facilitates movement of the polymer chains, thereby causing relaxation of the molded-in stresses, which, in turn, causes deformation of the devices 10,20. Thermal energy, or heat, is provided by the tissue and the water surrounding the anchor, due to both being at body temperature, or about 37° C. It is also believed that the incorporation of the glycolide provides an additional site for molecular motion and that the addition of water enhances the molecular motion of the glycolide unit relative to the lactide unit. This enhanced molecular motion results in a decrease in the relaxation temperature of the polymer material, which is normally about 50° C., and thus results in the change in shape of the material at about 37° C. The deformation of these devices 10,20 and the effect of this deformation, are more fully described below.

These devices 10,20 are used for the repair or remodeling of tissue. For example, the devices 10,20 may be used in treating a patient who has sustained an injury in which a soft tissue within their body has become detached (wholly or partially) from bone. The soft tissue may be a ligament, (e.g., the ACL), a tendon, a muscle, cartilage, or other soft or connective tissue.

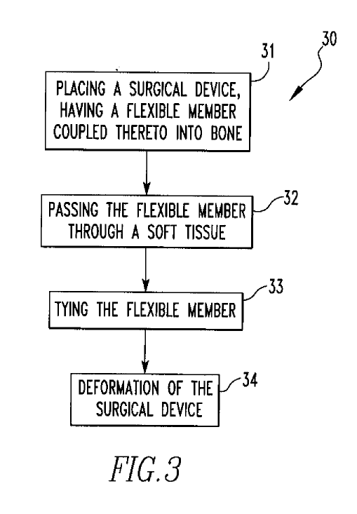

Accordingly, a method 30 used to repair a tissue via use of the devices 10,20 is shown in FIG. 3. First, a surgical device, having a flexible member coupled thereto, is placed in bone 31. The flexible members are then passed through the tissue that is located adjacent to the bone 32 and tied to secure the soft tissue to the bone 33. Deformation of the surgical device occurs upon insertion of the device into the bone 34, as described above. In order to insert the device into the bone, an opening may be formed in the bone that the device could be advanced into, via either rotary or axial advancement. Also, the flexible member is a suture material.

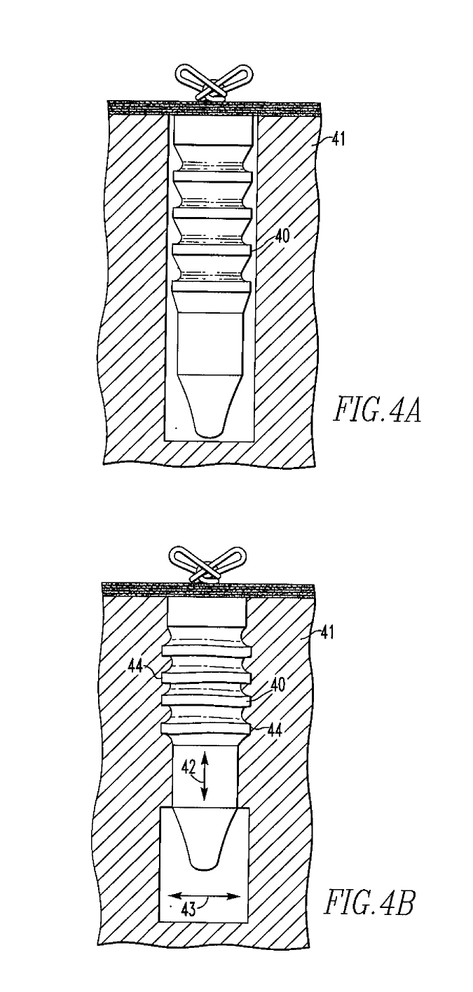

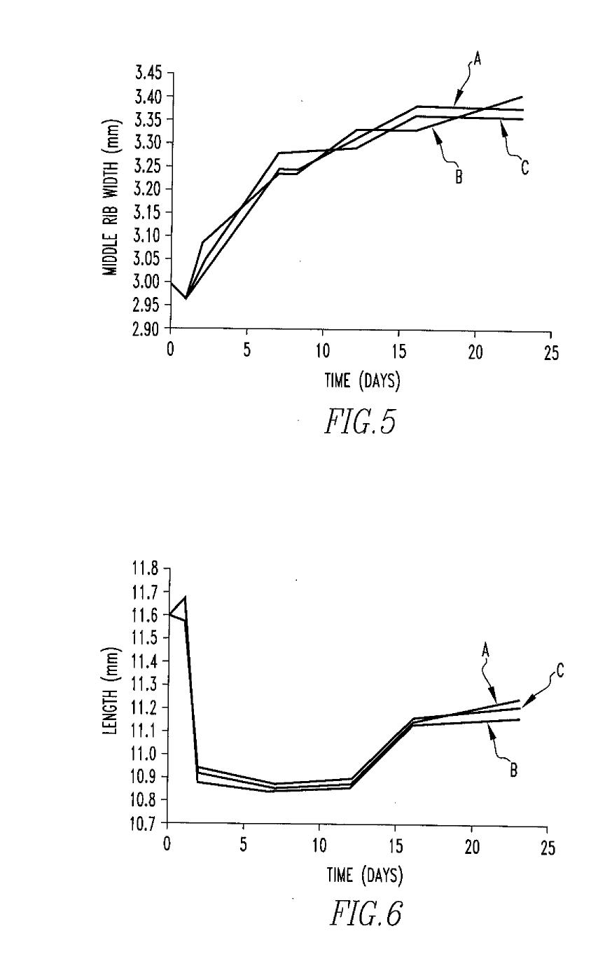

This change in shape is represented in FIGS. 4A and 4B. FIG. 4A shows the device 40 after it has been placed in the bone 41 and suture 45 has been passed through the tissue 46 that is located adjacent to the bone 41 and tied to secure the soft tissue 46 to the bone 41. FIG. 4B shows the device 40 after it has deformed. FIG. 4B illustrates that the device 40 has shrunk axially 42 and expanded radially 43, or in other words, there has been a decrease in the length of the device 40 and an increase in the width of the device 40. In addition, upon deformation, there is also an interlocking of the ribs 44 into the bone 41. In return, there is a substantial increase in the fixation strength of the device 40 that corresponds to the deformation, or increase in width and decrease in length, of the device 40. This enhanced fixation strength is described in further detail in the examples below. For simplicity purposes, the device 40 shown in FIGS. 4A and 4B is an anchor body having circumferential ribs, but may be an anchor body having screw threads or any other type of anchor body used to repair tissue. Also, for the purposes of this disclosure, deformation of the device 40 is not limited to an increase in width and a decrease in length. Rather, other types of deformation may occur. For example, the device may bend, but not necessarily increase in width. Factors that determine the type of deformation include, but are not limited to, material, mold design, and mold conditions.

Other methods of forming the devices include an extrusion process (e.g., a single screw, twin screw, disk, ram, or pulltrusion process); a different molding process, such as an intrusion, compression, or thermoforming process; a solvent based process (e.g., mixing or casting); a welding process (e.g., an ultrasonic or hermetic process); a polymerization process (e.g., reaction injection molding, bulk polymerization, and solvent polymerization); or by other methods (e.g., fiber spinning or electrospinning).

As copolymers, such as PLGA, degrade in vivo by hydrolysis into natural metabolic products, the devices or implants of the present disclosure are biocompatible and may also be referred to as bioabsorbable (i.e., as able to degrade over time in a biological environment, such as the human body, to compounds that are removed during normal metabolic processes). Moreover, devices fashioned with the present compositions can degrade over a period of time that allows a desirable shift in weight bearing from the device to the patient's own tissues.

The copolymer may include at least one rigid and/or one mobile polymer. An example of a mobile polymer includes polyethylene glycol and an example of a rigid polymer includes L-lactide or D-lactide. However, other mobile and rigid polymers known to those of ordinary skill in the art may be used. Mobile and rigid polymer components are used to modify the relaxation temperature and rates of the amorphous or semi-crystalline polymer material by modifying the crystallinity of the polymer material.

Furthermore, one or more hydrophilic materials may be included in the polymer matrix to accelerate water ingress and hence the relaxation rate of the polymer material. Examples of hydrophilic materials include polyethylene glycol. Other hydrophilic materials known to one of ordinary skill in the art may also be used.

The amorphous or semi-crystalline polymer composition, as described above, may include a porogen, such as sodium chloride, either alone or along with another filler material, such as the calcium carbonate described above. The porogen may then be washed out of the material leaving pores that will aid water penetration and hence accelerate the relaxation rate of the material. Porogens may be included in the amorphous or semi-crystalline material and washed out to leave pores before the material is oriented. Upon orientation of the material, channels will develop in the material, due to an increase in surface area, to aid in water penetration and relaxation rate. Since the rate of relaxation is dependent upon the diffusion rate of fluid into the polymer, the addition of these channels, pores, porogens, and hydrophilic units enhances the rate of relaxation of these materials. Alternatively, the porogens may be included in the device, such that upon placing the device in the body, the porogens dissolve out of the device, thereby leaving pores in the device. The effect of porogens, such as sodium chloride (NaCl), on the relaxation rate of the material, as compared to other fillers such as calcium carbonate (CaCO₃), is shown in FIG. 10. The effect of these porogens on the relaxation rate of the material may be varied by having a mixture of porogens with a range of solubilities and sizes. Other methods of varying the effect of these porogens, known to one of skill in the art, may also be used.

Other porogens known to one of ordinary skill in the art may also be used. Specifically, a porogen that causes an exothermic reaction, possibly upon dissolution of the porogen from the material and the reaction of the porogen with the in-vivo environment (i.e. water), would be useful in enhancing the relaxation rate of the polymer material. Examples of these porogens include, without limitation, lithium bromide, lithium iodide, calcium chloride, sodium iodide, magnesium sulphate, and calcium sulphate. In addition to leaving pores in the material, it is believed that the heat produced may diffuse through the material, thereby enhancing the mobility of the polymer chains and consequently further increasing the relaxation rate of the material. For the purposes of this disclosure, only porogens that release an amount of heat, which would not increase the temperature of the in-vivo environment to the glass-transition temperature of the material, would be used.

Inorganic particles, such as mineral particles, ceramic particles, and combinations thereof may also be included in the polymer materials to allow tailoring of the degradation and relaxation rates of the material. Examples of ceramic particles include calcium sulfate and calcium phosphate.

Rather than being a suture anchor, the devices can take the form of pins, rods, nails, screws, sutures, plates, sleeves for enhanced fixation of existing metal devices, plugs for cartilage repair, bone graft substitute, anchors, wedges, and other devices used for bone and tissue repair.

### Example One

Suture anchors of the present disclosure were sterilized using ethylene oxide. Three suture anchors were then placed in a phosphate buffered saline solution at 37° C., which stimulates the in vivo environment. Measurements of the anchor widths and lengths were taken at regular intervals and the results are shown in FIGS. 5 & 6, respectively. Over the course of about 3 weeks, the width of the suture anchors increased and the length decreased. A slight length increase was shown at 12 days. In both figures, results for the first, second, and third suture anchors are represented as A, B, & C, respectively.

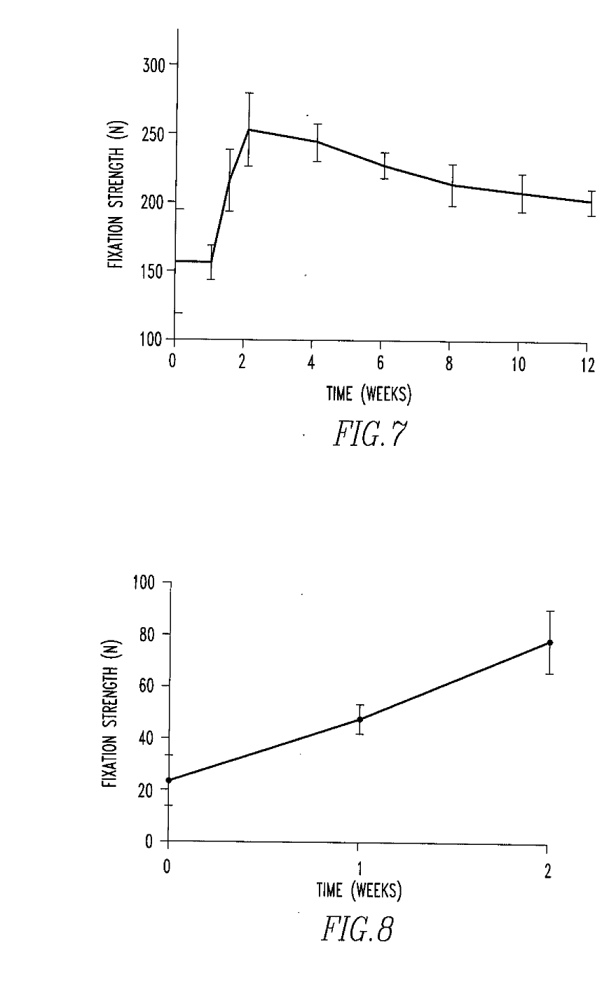

Anchors loaded with ultra high molecular weight polyethylene suture were evaluated for fixation strength in a simulated bone material over a period of time. A 2.6 mm hole was drilled into the center of a polyurethane simulated bone material with a density of 20 pcf, which represents good quality bone, and the anchor inserted into the hole. Each bone block, with inserted anchor, was placed in a jar and filled with phosphate buffered saline at 37° C., thereby simulating the in vivo environment. Ten samples were removed after one day for mechanical testing and then placed back in the solution and tested every two weeks for period of twelve weeks. The results are shown in FIG. 7. As can be seen, the fixation strength increased substantially by week 2 which corresponds to the increase in width demonstrated in FIG. 5 above. There was an increase of 96 N observed over this time period, which is equivalent to over 60% increase in fixation. Over the next ten weeks, there was a gradual decline in fixation strength, although still substantially above the initial fixation strength. This experiment was repeated in 10 pcf bone simulant material, which represents poor quality bone, for a period of two weeks. The results are demonstrated in FIG. 8. During this time, an increase in fixation strength of 55N, or 230%, was observed.

### Example Two

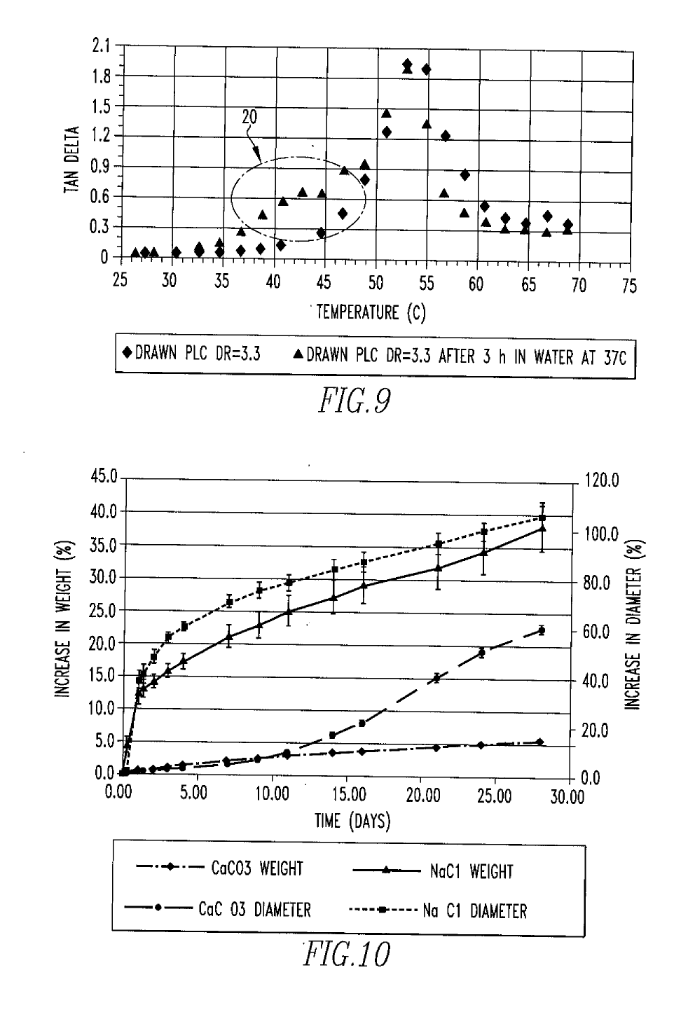

Resorbable, oriented amorphous zone drawn fibers of Poly (D,L lactide-co-glycolide) and calcium carbonate were placed into water at a temperature of 37° C. for 3 hours. The ratio of lactide:glycolide was 85:15 and the calcium carbonate was present at between about 30% to about 40% by weight of the polymer composition. The fibers were removed, surface dried, and analyzed using dynamic mechanical thermal analysis (DMTA). The data from the DMTA was compared to DMTA data of Poly (D,L lactide-co-glycolide)+calcium carbonate fibers that had not been placed in water. FIG. 9 shows the results of this comparison. In FIG. 9, the triangle represents the drawn fibers that were placed in water for 3 hours at 37° C. and the diamond represents the drawn fibers that had not been placed in water. The draw ratio for both fibers was 3.3. The draw ratio is a measure of the degree of stretching during the orientation of a fiber, expressed as the ratio of the cross-sectional area of the undrawn material to that of the drawn material. The DMTA analysis was carried out at 1 Hz with a dynamic strain of 0.05%. The time at each temperature was chosen as 20 seconds and sampling was taken in steps of 2° C. from 26° C. to 70° C. From FIG. 9, it is clear that there is a small relaxation peak 50 at a lower temperature from the main peak 60 for the sample immersed in water, which, as mentioned above, is indicative of the water causing a decrease in the normal relaxation temperature of the material due to enhancement of the molecular mobility of the polymer chains.

### Example Three

A hole having a diameter of about 8.5 mm was drilled into a sawbone and a die drawn plug constructed of Poly (D,L lactide-co-glycolide) and calcium carbonate was inserted. The fixation strength of the plug was determined by using a push-out test. The push-out force of an initial dry plug was measured using an Instron and was found to be 0 N. The Instron was operated at 1 mm/min. The plug was immersed in water at 37° C. and soaked for 9 days. The push-out force of the plug was then measured and was found to be about 1700 N. The relaxation of the oriented network was responsible for the tight fit and enhanced fixation strength.

### Example Four

Two poly (D,L-lactide-co-glycolide) 85:15 based die drawn rods were produced. One included 35% w/w CaCO₃ filler, while the other included 35% w/w NaCl filler. The polymer and filler were combined using a twin-screw extruder and the resulting pellets were molded to produce 30 mm diameter rods. The rods were die-drawn at 75° C. through a 15 mm diameter die at 30 mm/min and at a draw ratio of 3.5. Six 3 cm long samples of each type of rod were weighed and the diameters were measured. The rods were subsequently placed in 8 oz glass jars containing phosphate buffered saline and then placed in an incubator at 37° C. Periodically the samples were removed from the buffer, wiped dry, weighed, measured, and re-placed in the buffer and then returned to the incubator. The increases in weight and diameter of the rods are shown in FIG. 10.

FIG. 10 shows that the highly porous NaCl containing rod absorbed water and started to expand in diameter much more rapidly than the CaCO₃ containing rod. No significant change in diameter was observed in the NaCl containing rods after 0.29 days, yet by 1.07 days its diameter had increased by 38% rising to 40.6% after 1.33 days. The CaCO₃ containing rod took 7 days to increase in diameter by 4.07% and 21 days to achieve a 40.09% increase. Hence, it can be concluded that the incorporation of pores or porogens, such as sodium chloride, may significantly enhance the ingress of water into the polymer material and hence the relaxation rate of the polymer material, thereby leading to an accelerated increase in diameter of the rod.

The increase in the relaxation rate of the material having the NaCl as compared to the material having the CaCO₃ may be defined by the following equation:

$$\frac{slope of line for porogen containing material per day}{\begin{matrix}slope of line for calcium \\ carbonate containing material per day\end{matrix} }$$

The lines referred to in the equation are the lines, in FIG. 10, that refer to the diameters of the sodium chloride and the calcium carbonate. The slope of the line for the porogen containing material per day was about 35.5% and the slope of the line for the calcium carbonate containing material per day was about 0.87%. Inserting these values into the above equation shows that incorporating 35% NaCl into the material increased the relaxation rate of the material by 40 times compared to the calcium carbonate containing material. The gradient of the slope may be dependent on a variety of factors, including but not limited to, the form of the material.

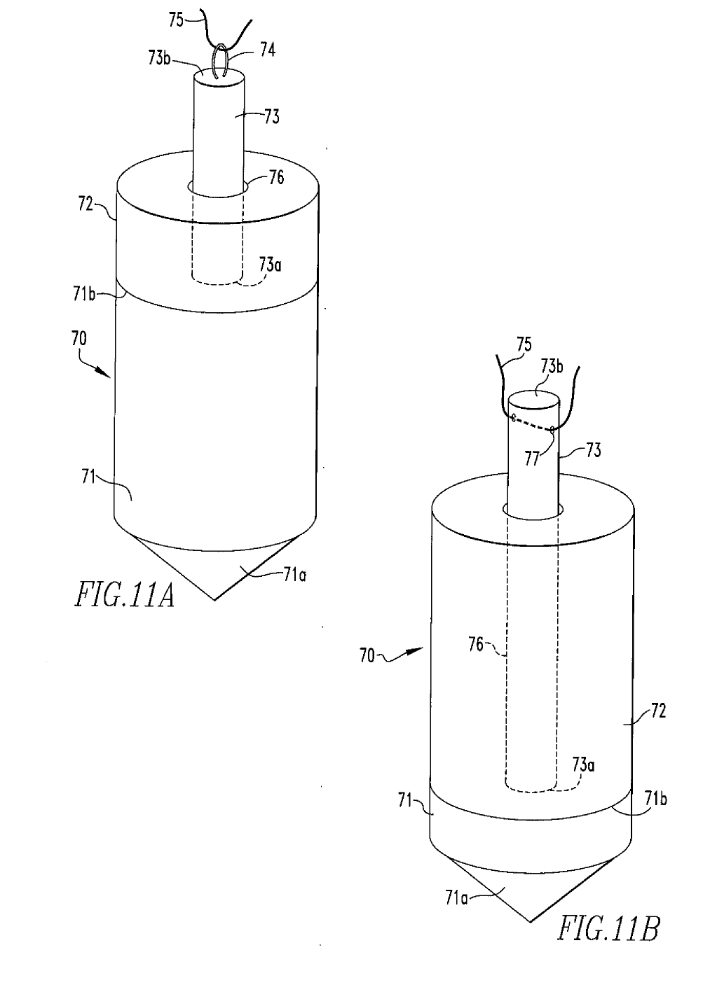

FIG. 11A shows a suture anchor 70 including a first component 71 having a pointed distal end 71a, a proximal end 71b, and a shaft 73 coupled to the proximal end 71b. The shaft 73 includes a distal end 73a and a proximal end 73b. A second component 72 is coupled to the first component 71. The second component 72, which includes a through hole 76, is coupled to the first component 71 such that the shaft 73 extends through the hole 76. An eyelet 74 is coupled to the proximal end 73b of the shaft 73 and a suture is coupled to the anchor 70 via the eyelet 74.

FIG. 11B shows a suture anchor 70 similar to the suture anchor 70 shown in FIG. 11A. However, the second component 72, through hole 76, and shaft 73 of FIG. 11B are longer the second component 72, through hole 76, and shaft 73 of FIG. 11A and the suture 75 is disposed within a groove 77 of the shaft 73, rather than being disposed within an eyelet, as in FIG. 11A. In addition, the first component 71 of FIG. 11B is shorter than the first component 71 of FIG. 11A. For the purposes of this disclosure, the groove 77 is located on the shaft, may be located anywhere on the anchor 70. Also for the purposes of this disclosure, the components 71,72 may include features, such as threads, barbs, ribs, or other features known to one of skill in the art, on an outer surface of the components 71,72, such that the features may allow for increased fixation of the suture anchor 70 to bone when the anchor 70 is placed in bone.

Component 72 may be made from a highly orientated polymer material and component 71 may be made from a low orientated polymer material or vice versa. Components having high or low orientations may be made via a die drawing process, whereby drawing a polymer material at a draw ratio of below 2 would produce a low orientated component and drawing a polymer material at a draw ratio of above 2 would produce a highly orientated component. Alternatively, different processes may be used to make the high and low oriented components. For example, highly oriented component 72 may be made via a die drawing process and low oriented component 71 may be made via an injection molding process Having a low or high orientation may be a good indicator of the deformation capability, particularly the radial expansion capability, of the material. For example, a polymer material having a low orientation would not expand as much as a polymer material having a high orientation. Therefore, when the anchor 70 is placed in bone, fixation of the anchor 70 to the bone may be stronger in one area of the anchor 70 than in another. Other methods of providing the components 71,72 with an orientation, such as hydrostatic extrusion, roll drawing, and other methods known to one of skill in the art, may also be used

The polymer material of components 71,72 may include the calcium carbonate containing polymer material and/or the porogen containing polymer material described above. For example, components 72 and 71 of FIG. 11A may be made from the porogen containing material and the calcium carbonate containing material, respectively. In this instance, and based on examples 1-4 above, component 72 may have a higher rate of relaxation than component 71. As mentioned above, the rate of relaxation for the porogen containing material may vary based on the amount and type of porogen that is used.

Component 72 may be coupled to component 71 mechanically by press fitting the through hole 76 over the shaft 73 or via rotary advancement by having threads on the shaft 73 and on an inner wall of the through hole 76 that are configured to engage each other when component 72 is disposed on component 71. Other mechanical means are also within the scope of this disclosure. Alternatively, component 72 may be coupled to component 71 chemically via the use of a biocompatible adhesive or solvent or by melting or welding the component 72 to component 71. Other methods of coupling are within the scope of the disclosure. The components 71,72 may be made via a method described above or by any other method known to one of skill in the art. In addition, the holes 76,77 may be made by drilling or another method known to one of skill in the art. Also, each of components 71,72 may be made from a single piece of material, as shown in FIGS. 11A-11B, or several pieces of material with each piece being the same material or different material. Furthermore, the shape of shaft 73 and hole 76 may be other than circular.

Use of the anchors 70 may occur in the same manner described above and shown in FIG. 3. The anchor 70 may be placed in the bone such that the entire anchor 70 (including the eyelet in FIG. 11A) is below the surface of the bone or the proximal end 73b of the shaft 73 is flush with the surface of the bone.

As described above, the incorporation of glycolide to the polymer material results in a decrease in the relaxation temperature of the polymer material, which is normally about 50° C., and thus results in the change in shape of the material at body temperature, or about 37° C. Other materials that would cause a decrease in the relaxation temperature of the polymer material of this disclosure, such as, but not limited to, caprolactone and trimethylene carbonate, may also be used.

As various modifications could be made to the exemplary embodiments, as described above with reference to the corresponding illustrations, without departing from the scope of the disclosure, it is intended that all matter contained in the foregoing description and shown in the accompanying drawings shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. A surgical device comprising:
an anchor body including an opening, the anchor body having a copolymer composition including polylactide-co-glycolide and calcium carbonate, wherein the calcium carbonate comprises more than 30% but less than 40% of the weight of the composition; and
a flexible member passing through the opening, wherein deformation of the device occurs at body temperature.

2. The surgical device of claim 1 wherein the opening comprises a through hole.

3. The surgical device of claim 1 wherein the anchor body is configured for rotary advancement into a target tissue.

4. The surgical device of claim 1 wherein the anchor body includes screw threads.

5. The surgical device of claim 1 wherein the anchor body is configured for axially oriented advancement into a target tissue.

6. The surgical device of claim 1 wherein the anchor body includes circumferential ribs.

7. The surgical device of claim 1 wherein the device is injection molded.

8. The surgical device of claim 1 wherein deformation of the device occurs at about 37° C.

9. The surgical device of claim 1 wherein the device is bioabsorbable.

10. A method for repairing a soft tissue comprising:
placing a surgical device in bone, the surgical device having a flexible member coupled thereto and having a copolymer composition including polylactide-co-glycolide and calcium carbonate, wherein the calcium carbonate comprises more than 30% but less than 40% of the weight of the composition;
passing the flexible member through a soft tissue located adjacent to the bone; and
tying the flexible member to secure the soft tissue to the bone,
wherein deformation of the surgical device occurs at body temperature after placement of the device in the bone.

11. The method of claim 10 wherein the surgical device comprises a suture anchor.

12. The method of claim 10 wherein deformation of the device provides an increase in fixation of the device to the bone.

13. The method of claim 12 wherein the increase in fixation comprises an increase in fixation strength of the device of between about 50% to about 200%.

14. The method of claim 10 wherein deformation of the device provides an increase in width of the device and a decrease in length of the device.

15. An oriented polymer material including a copolymer composition having a polylactide-co-glycolide and calcium carbonate, the calcium carbonate comprising more than 30% but less than 40% of the weight of the composition, wherein the material changes shape upon introduction to an environment having a temperature that is lower than a relaxation temperature of the material.

16. The polymer material of claim 15 wherein the polylactide-co-glycolide comprises poly(D,L lactide-co-glycolide).

17. The polymer material of claim 15 wherein the copolymer includes at least one mobile polymer.

18. The polymer composition of claim 17 wherein the copolymer further comprises at least one rigid polymer.

19. The polymer material of claim 15 wherein the copolymer includes at least one rigid polymer and one mobile polymer.

20. The polymer material as in claim 17 or 19 wherein the mobile polymer comprises polyethylene glycol.

21. The polymer material as in claim 18 dr 19 wherein the rigid polymer is selected from a group consisting essentially of L-lactide, D-lactide, and D,L-lactide.

22. The polymer material of claim 15 wherein the material includes a porogen.

23. The polymer material of claim 22 wherein the porogen includes sodium chloride.

24. The polymer material of claim 15 wherein the temperature of the environment is about 37° C.

25. The polymer material of claim 15 wherein the temperature of the environment comprises body temperature.

26. The polymer material of claim 15 wherein the relaxation temperature is about 50° C.

27. The polymer material of claim 15 wherein the polymer material comprises a fixation strength of above 500 N.

28. A surgical device comprising a copolymer composition including polylactide-co-glycolide and a porogen.

29. The surgical device of claim 28 wherein the surgical device comprises an oriented polymer material.

30. The surgical device of claim 28 wherein the porogen includes sodium chloride.

31. The surgical device of claim 28 wherein the porogen is selected from a group consisting essentially of lithium bromide, lithium iodide, calcium chloride, sodium iodide, magnesium sulphate, and calcium sulphate.

32. The surgical device of claim 28 wherein the surgical device is selected from a group consisting essentially of pins, rods, nails, screws, sutures, plates, anchors, and wedges.

33. A surgical device comprising a first component including a shaft, a second component coupled to the first component, and a flexible member coupled to the shaft, wherein the first component is an injection molded component and the second component includes an oriented polymer material.

34. The surgical device of claim 33 wherein the flexible member is coupled to the shaft via an eyelet, the eyelet coupled to the shaft.

35. The surgical device of claim 33 wherein the flexible member is coupled to the shaft via an opening in the shaft.

36. The surgical device of claim 33 wherein the first component includes a copolymer composition having polylactide-co-glycolide and calcium carbonate and the second component includes a copolymer composition having polylactide-co-glycolide and a porogen.

37. A surgical device comprising a first component including a shaft and second component coupled to the first component, wherein the first component includes a copolymer composition having polylactide-co-glycolide and calcium carbonate and the second component includes a copolymer composition having polylactide-co-glycolide and a porogen.

38. The surgical device in any of claims 29 or 33 wherein the oriented polymer material is made by a process selected from a group consisting essentially of die drawing, hydrostatic extrusion, and roll drawing.
